(19) United States
(12) Plant Patent Application Publication
(10) Pub. No.: US 2007/0094758 P1
(43) Pub. Date: Apr. 26, 2007

(54) 'Poulpal026'

(76) Inventor: Mogens N. Olesen, Fredensborg (DK)

Correspondence Address: POULSEN ROSER APS, 620 S. FRONT STREET, CENTRAL POINT, OR, 97502, US

(21) Appl. No.: 11/528,940

(22) Filed: Sep. 27, 2006

(30) Foreign Application Priority Data
Oct. 25, 2005 (EP) 2005/2031

(51) Int. Cl. A01H 5/00

(52) U.S. Cl. PLT/118

(57) ABSTRACT

A new miniature rose plant that has abundant, deep yellow flowers and attractive foliage. The variety successfully propagates from softwood cuttings and is suitable for year-round production in commercial glasshouses. This new and distinct variety has shown to be uniform and stable in the resulting generations from asexual propagation.

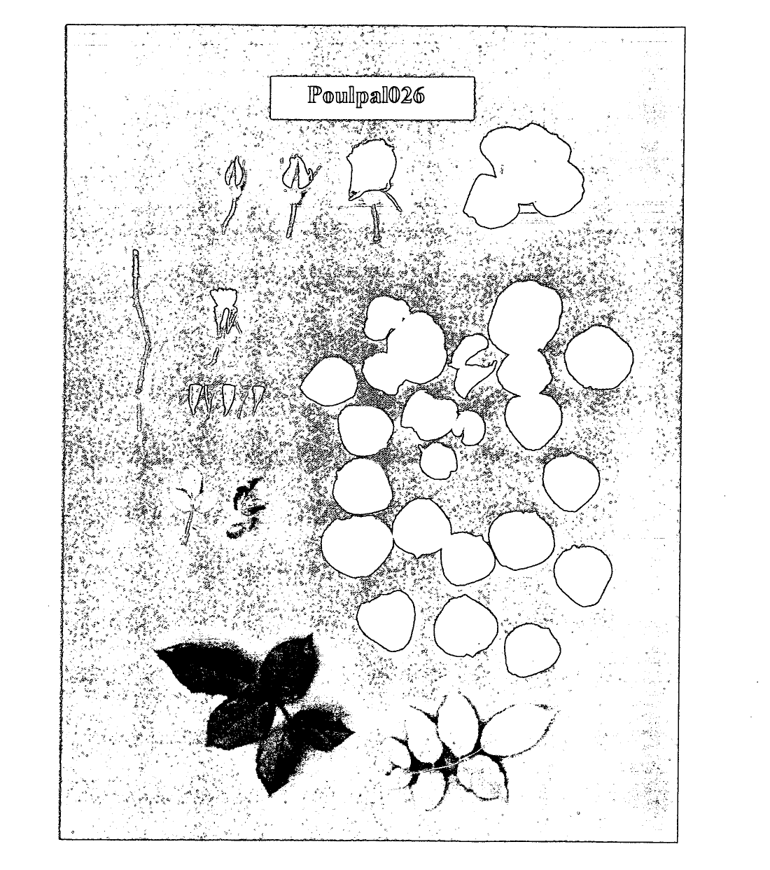

### BOTANICAL DESIGNATION

Rosa hybrid

### VARIETY DENOMINATION

‘Poulpal026’

### SUMMARY OF THE INVENTION

The present discovery constitutes a new and distinct variety of miniature rose plant which was discovered in a cultivated area. The new rose variety resulted from a naturally occurring mutation of unknown causation on a branch of ‘Poulac002’, U.S. Plant Pat. No. 13,277. The resulting mutation was selected and evaluations were conducted on the resulting rose plants in a controlled environment.

The rose plant of the present discovery has a unique combination of characteristics which are outstanding in the new variety and which distinguish it from the original rose ‘Poulac002’ as well as all other varieties which we are aware of. For example, open flowers of ‘Poulac002’ have a general tonality of Yellow Group 13B. No change in the general tonality at the end of the 4^{th} day. Afterwards, general tonality is Yellow-Orange Group 14C. Flowers of ‘Poulpal026’ have a general tonality of Yellow Group 12A with light intonations of Yellow Group 13A to 13B. Afterwards, general tonality is Yellow Group 10B to Yellow Group 12B.

The criteria used to evaluate the claimed plant were selected in order to introduce a distinct variety for garden use with unique qualities, such as:
1. Uniform and abundant deep yellow flowers;
2. Vigorous, but compact growth when propagated both as a budded rose and on its own roots;
3. Exceptional disease resistance; and
4. Durable flowers and foliage.

This combination of qualities is not present in previously available commercial cultivars of this type, known to the inventor, and distinguish ‘Poulpal026’ from all other varieties of which we are aware.

Asexual reproduction of ‘Poulpal026’ by vegetative cuttings was first done by Mogens N. Olesen in Fredensborg, Denmark, in July of 1996. This initial and other subsequent propagations have demonstrated that the characteristics of ‘Poulpal026’ are stable and reproduced true to type in successive generations of asexual reproduction.

### BRIEF DESCRIPTION OF THE DRAWING

The accompanying color illustration shows as true as is reasonably possible to obtain in color photographs of this type, the typical characteristics of the buds, flowers, leaves, and stems of ‘Poulpal026’. Specifically illustrated in the drawing are flower buds, open flowers, sexual reproductive flower parts, juvenile and mature foliage, and a portion of the stem without leaves.

### DETAILED DESCRIPTION OF THE VARIETY

The following is a description of ‘Poulpal026’, as observed in its growth in a glasshouse in Fredensborg, Denmark. Observed plants are 4 months of age and were cultivated in 12 cm pots. Color references are made using The Royal Horticultural Society (London, England) Colour Chart, 2001, except where common terms of color are used.

For a comparison, several physical characteristics of the rose variety ‘Poulac002’, U.S. Plant Pat. No. 13,277, are compared to ‘Poulpal026’ in Chart 1.

CHART 1

| | ‘Poulpal026’ | ‘Poulac002’ |
|---|---|---|
| Flower | Average flower | 45 to 55 mm. |
| diameter | diameter is 65 | |
| | to 80 mm. | |
| Upper surface | Yellow Group 10A | Yellow-Orange |
| of petals on | to 12B. | Group 15A. |
| open flower. | | |
| Flower bud | As sepals unfold, | As sepals unfold, |
| color | petals are Yellow-Green | Orange-Red Group |
| character- | Group 150B with distinct | 31B. Guard petals |
| istics, | intonations of Yellow- | have variable |
| | Green Group N144A. As | coloration of |
| | the flower bud further | Yellow-Green Group |
| | develops, petals become | 146C and Red Group |
| | Yellow Group 13A to | 50A. Yellow-Orange |
| | Yellow-Orange Group | Group 21B at ¼ |
| | 16A. | opening. |

Flower and flower bud:
Blooming habit: Continuous.
Flower bud:
   Size.—Upon opening, 25 to 30 mm in length from base of receptacle to end of bud. 13 mm in diameter.
   Bud form.—Ovate to urceolate.
   Bud color.—As sepals unfold, petals are Yellow-Green Group 150B with distinct intonations of Yellow-Green Group N144A. As the flower bud further develops, petals become Yellow Group 13A to Yellow-Orange Group 16A.
   Sepals.—Upper Surface: Color: Green Group 138B. Texture: Strongly Pubescent. Lower Surface: Color: Yellow-Green Group 144A with anthocyanin the color of Greyed-Orange N170A at the apex. Texture: Smooth. Shape: Apex: Cirrhose. Base: Flat at union with receptacle. Margins: Margins have weak foliaceous appendages on three of the five sepals. Stipitate glands are abundant, protruding to 1 mm on occasion. Size: 25 mm long by 6 mm wide.
   Receptacle.—Surface Texture: Smooth. Shape: Pear to urn shaped. Size: 8 mm in height by 8 mm wide. Color: Yellow-Green Group 144A.
   Pedicel.—Surface: Slightly rough with stipitate glands. Length: 25 to 30 mm. Diameter: Normally 3 mm. Color: Yellow-Green Group 144A. Strength: Medium strength.
   Borne.—Normally singly.
Flower bloom:
   Fragrance.—Medium floral scent.
   Duration.—As a pot plant, flowers last from 18 to 20 days. Petals fall cleanly away from plant after maturity.
   Size.—Average flower diameter is 65 to 80 mm. Depth is normally 30 to 35 mm.
   Form.—Flowers are generally cupped, while the center tends to retain a globular form.
   Shape of flower, side view.—Upon opening, the upper portion is a flattened convex. The lower portion is flat to somewhat convex. After opening, the upper portion is flattened convex. The lower portion is convex.
Petalage: There are normally 25 petals, 3 to 7 of which are petaloids.
Color:
   Upon opening, petals.—Outermost and innermost petals: Upper Surface: Yellow Group 12A. Lower Surface: Yellow Group 12A with intonations of Yellow Group 13B.
   Upon opening, basal petal spots.—No distinctive coloration at petal base observed.
   After opening, petals.—Outermost and innermost petals: Upper Surface: Yellow Group 10A to 12B. Lower Surface: Yellow Group 10A.
   After opening, basal petal spots.—No distinctive coloration at petal base observed.
General tonality: On open flower Yellow Group 12A with light intonations of Yellow Group 13A to 13B. Afterwards, general tonality is Yellow Group 10B to Yellow Group 12B.
Petals:
   Petal reflex.—Slightly reflexed.
   Petal margin.—Entire with a point at the center of the apex. Medium undulations of margin observed.
   Shape.—Generally broadly elliptical. Base: Acute. Apex: Cuspidate.
   Size.—Outer petals are generally 45 mm long by 45 mm wide. Inner petals are generally 35 mm long by 35 mm wide.
   Texture.—Smooth.
   Thickness.—Somewhat above average.
Petaloids:
   Quantity.—Normally, there are 3 to 7.
   Size.—20 to 25 mm long; 20 to 25 mm wide.
   Shape.—Irregular. The apex is rounded with an occasional cleft in the margin. The base is acute.
   Color.—Upper Surface: Yellow Group 10A to 12B. Lower Surface: Yellow Group 10A.
Reproductive organs:
   Pollen.—None observed.
   Anthers.—Size: 3 mm long. Color: Yellow Group 8C and Yellow-Orange Group 14A. Quantity: 110 to 120.
   Filaments.—Color: Yellow-Orange Group 17A. Length: 7 to 10 mm.
   Pistils.—Length: 2 to 4 mm long. Quantity: 50 to 60.
   Stigmas.—Slightly inferior relative to the length of the filaments and the height of the anthers. Color: Greyed-Orange Group 164D.
   Styles.—Color: Red Group 50A.
   Seed formation.—Not observed.
Plant:
Plant growth: When grown as a 12 cm pot plant on its own roots, the average height of the plant itself is 20 to 25 cm. The average width is 20 cm.
Stems:
   Colo.—Young wood: Yellow-Green Group 144B. Older wood: Yellow-Green Group 146B.
   Internodal distance.—25 to 29 mm.
   Length of stems.—From the base of the stem to the flowering portion, 15 to 18 cm.
   Diameter: 3 to 4 mm.
   Surface texture.—Young wood: Smooth. Older wood: Smooth.
Prickles:
   Incidence.—15 to 18 per 10 cm of stem.
   Size.—Average length is 6 to 9 mm.
   Color.—Juvenile prickles are Yellow-Green Group 145B. Mature prickles are Greyed-Red Group 182C.
   Shape.—Linear.
Plant foliage: Normal number of leaflets on normal leaves in middle of the stem: 7 leaflets.
   Compound Leaf size.—110 to 140 mm (l)×90 to 105 mm (w).
   Quantity.—4 leaves per 10 cm of stem.
   Color.—Juvenile foliage: Upper Leaf Surface: Yellow-Green Group 146B. Lower Leaf Surface: Yellow-Green Group 146C. Anthocyanin: Greyed-Purple Group 185A generalized. Mature foliage: Upper Leaf Surface: Yellow-Green Group 147A. Lower Leaf Surface: Yellow-Green Group 147B to 147C.
Plant leaves and leaflets:
   Stipules.—Size: 12 mm in length. Shape: Linear, slightly broad based with outward extending apecies. Margins: Finely serrated with few stipitate glands. Color: Yellow-Green Group 146A.
   Petiole.—Length: 10 to 30 mm. Diameter is 1.5 mm.
   Color.—Yellow-Green Group 146A at the upper portion. The lower portion is Yellow-Green Group 146B.
   Anthocyanin.—Greyed-Purple Group 187A at margins.
   Lower surface.—Few small prickles observed.
Rachis:
   Size.—40 to 50 mm in length.
   Color.—Upper surface is Yellow-Green Group 146A to 146B with pigments the color of Greyed-Purple Group 187A at the margins. The lower surface is Yellow-Green Group 146B.
   Upper surface.—Few stipitate glands observed.
   Lower surface.—Small prickles observed.
Leaflet:
   Size.—55 to 65 mm in length by 29 to 40 mm wide.
   Margin.—Serrated.
   General shape.—Ovate.
   Apex shape.—Acuminate.
   Base shape.—Obtuse.
   Texture.—Smooth.
   Thickness.—Average.
   Arrangement.—Odd pinnate.
   Venation.—Reticulate.
   Leaf gloss.—Moderately glossy.
Disease resistance: Average resistance to powdery and downy mildew, black spot, and Botrytis under normal growing conditions in Fredensborg, Denmark.

1. A new and distinct variety of rose plant of the miniature class named ‘Poulpal026’, substantially as illustrated and described herein, due to its abundant, deep yellow flowers, vigorous growth, compact habit, suitability for production from softwood cuttings in pots, and durable flowers and foliage that make the variety suitable for distribution in the floral industry.
